(19) United States
(12) Patent Application Publication
(10) Pub. No.: US 2008/0103058 A1
(43) Pub. Date: May 1, 2008

(54) Molecules and methods for nucleic acid sequencing

(75) Inventor: Suhaib Siddiqi, Burlington, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: Helicos BioSciences Corporation, Cambridge, MA (US)

(21) Appl. No.: 11/928,618

(22) Filed: Oct. 30, 2007

Related U.S. Application Data
(63) Continuation of application No. 11/643,742, filed on Dec. 20, 2006.
Application No. 11/643,742 is a continuation of application No. 11/412,569, filed on Apr. 26, 2006, now abandoned.

(51) Int. Cl. C40B 30/04; C07H 21/04

(52) U.S. Cl. 506/9; 536/26.2; 536/22.1

(57) ABSTRACT

The invention provides molecules and methods for nucleic acid synthesis reactions useful in sequencing-by-synthesis processes.

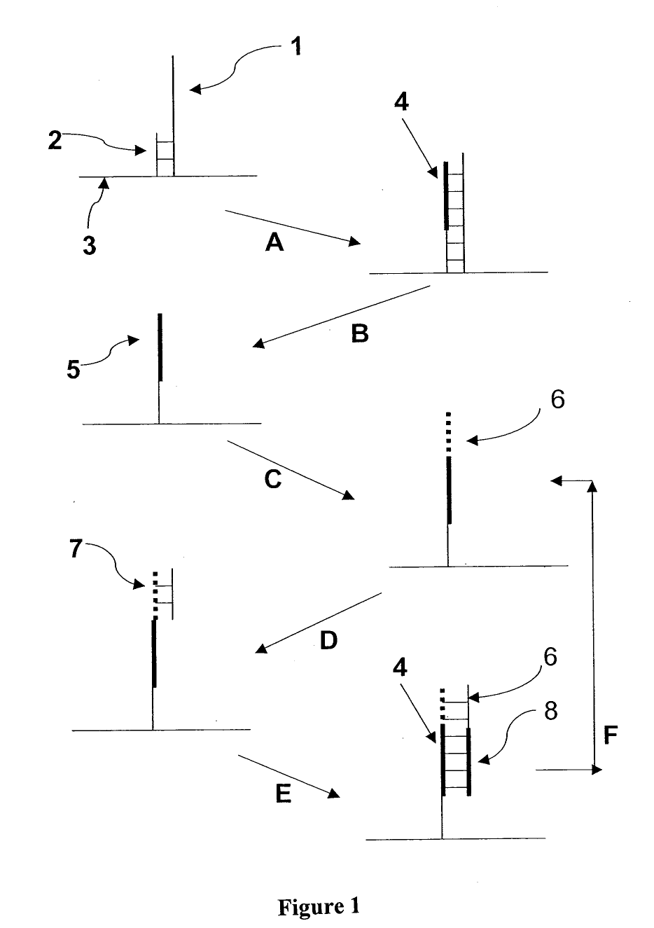

### RELATED APPLICATIONS

This application is a continuation of U.S. application Ser. No. 11/412,569 filed Apr. 26, 2006, pending, the entire contents of which is expressly incorporated herein by reference.

### TECHNICAL FIELD OF THE INVENTION

The invention generally relates to molecules and methods for nucleic acid sequencing reactions.

### BACKGROUND OF THE INVENTION

In vitro nucleic acid sequencing is a foundational research and commercial tool. In a template-dependent nucleic acid sequencing reaction, the sequential addition of nucleotides is catalyzed by a nucleic acid polymerase. Depending on the template and the nature of the reaction, the nucleic acid polymerase may be a DNA polymerase, an RNA polymerase, a reverse transcriptase, or a modified polymerase.

Single molecule sequencing techniques allow the evaluation of individual nucleic acid molecules in order to identify changes and/or differences affecting genomic function. In single molecule techniques, a nucleic acid fragment is attached to a solid support such that it is individually optically-resolvable. Sequencing is conducted using the fragments as templates. Sequencing events are detected and correlated to the individual strands. See Braslavsky et al., Proc. Natl. Acad. Sci., 100: 3960-64 (2003), incorporated by reference herein.

In the template-dependent sequencing, nucleic acids added to the 3′ terminus of a primer associated with each template. The added nucleic acids typically contain a label that allows a step-wise observation of incorporation. Manipulation of the label and functional groups to control the primer extension are desirable ways to control the reaction. For example, one problem is the presence of homopolymeric sequences (i.e., base repeats). The number of bases in a homopolymer is often of diagnostic or clinical significance. Thus, it is often desirable to add only one nucleotide at a time to the 3′ terminus of the primer. Without adequate control of nucleotide addition, it may be difficult to determine the number of nucleotides in a homopolymeric run.

There is, therefore, a need in the art for improved methods for controlling nucleic acid sequencing reactions, especially in the context of single molecule sequencing.

### SUMMARY OF THE INVENTION

The invention improves the efficiency of nucleic acid sequencing reactions. The invention solves the problem of controlling base addition in a sequencing-by-synthesis reaction by providing nucleotide analogs that allow control of the number of nucleic acids added to the primer. Analogs and methods of the invention, allow the addition of one appropriate (i.e., Watson-Crick base-paired) nucleotide to the 3′ terminus of the primer followed by reversible inhibition of further additions to the primer. Upon removal of the inhibition, sequencing continues one base at a time. The invention allows, among other things, the ability to count base additions in a homopolymeric region.

In a specific embodiment the invention relates to compounds of formula (I) or (II) (e.g., nucleotide(s)/nucleotide analog(s)) and methods for their use.

One aspect is a molecule of formula (I), or salt, hydrate or solvate thereof: 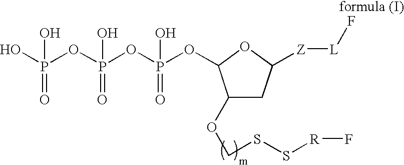
wherein,

Z is a purine, pyrimidine or analog thereof,

L is a linker;

Each F is independently an optically-detectable label;

R is alkyl; and

m is an integer greater than 1;
another aspect is a molecule of formula (II), or salt, hydrate or solvate thereof: 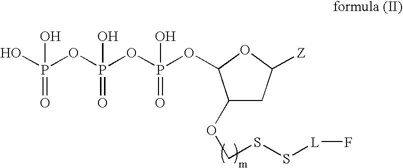
wherein,

Z is a purine, pyrimidine or analog thereof,

L is a linker;

F is an optically-detectable label; and

m is an integer greater than 1.

Other aspects are compounds of the formulae herein, wherein the linker comprises an alkynyl group; wherein the linker comprises the structure: 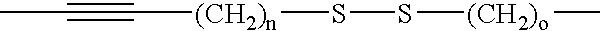
wherein, n is an integer 1-7 inclusive; and o is an integer 1-7 inclusive; wherein each F is independently a fluorescent label; wherein each F is independently cyanin-3 or cyanin-5; wherein R is an alkyl having from about 1 to about 12 carbon atoms; wherein the purine is adenine, guanine, or analog thereof; wherein the pyrimidine is cytosine, thymidine, uracil, or analogs thereof; wherein the linker comprises an alkynyl group; or wherein m is 2.

Another aspect is a method for synthesizing a nucleic acid analog comprising contacting a nucleic acid sequence with a compound of formula (I) or formula (II), or salt, hydrate or solvate thereof.

The invention also relates to methods of performing nucleic acid sequencing. Another aspect is a method for sequencing a nucleic acid template comprising: (a) exposing a nucleic acid duplex comprising a template nucleic acid hybridized to a primer nucleic acid to a plurality of molecules of a compound according to any of the formulae herein under conditions that allow the molecule to be incorporated into the 3′-terminus of the primer and to engage in complementary base pairing with a nucleotide in the template. The method can further comprise: (b) removing unincorporated molecules of the compound of any of the formulae herein; (c) observing a label associated with the compound of any of the formulae herein; (d) removing the label; (e) modifying the incorporated molecule to generate a free 3′-hydroxy group, and (f) repeating steps (a) to (e). In other aspects the method is that: further comprising repeating step (f); wherein step (b) comprises exposing the duplex to an agent capable of reducing disulfide bonds; further comprising the step of identifying the molecule incorporated into the primer; wherein step (d) comprises exposing the duplex to an agent capable of reducing disulfide bonds; wherein step (e) comprises exposing the duplex to an agent capable of reducing disulfide bonds; wherein steps (d) and (e) are performed simultaneously; wherein the agent is tris(2-carboxyethyl)phosphine hydrochloride (TCEP-HCl); wherein the reduction of the disulfide bond is performed at about pH 7.0 or greater; wherein the reduction of the disulfide bond is performed at about pH 9.0 or greater; wherein the reduction of the disulfide bond is performed at about 25° C. or greater; wherein the reduction of the disulfide bond is performed at about 37° C. or greater; or wherein the reduction of the disulfide bond is performed at about 50° C. or greater.

According to the invention, a polymerization reaction is conducted on a nucleic acid duplex that comprises a primer hybridized to a template nucleic acid. The reaction is conducted in the presence of a polymerase, and at least one nucleotide comprising a detectable label. If the nucleotide is complementary to the next available nucleotide in the template, it is added to the primer by the polymerase. The added nucleotide is detected and the reaction is then repeated at least once. Thus, the primer is extended by one or more nucleotides corresponding to sequence that is complementary to at least a portion of the template. The template is then optionally removed from the duplex, leaving the extended primer.

In one embodiment, one or more primer/template duplexes are bound to a solid support such that a least some of the duplexes are individually optically resolvable. The duplexes are exposed to a polymerase, and at least one detectably-labeled inhibitory nucleotide according to the invention under conditions sufficient for template-dependent nucleotide addition to the primer. Unincorporated labeled nucleotides are optionally removed. The incorporation of the labeled nucleotide is detected, thereby identifying the added nucleotide and the complementary template nucleotide. The inhibition is removed, either before, during, or after detection, and the base addition, washing, and identification steps can be serially repeated. As a result, primers are extended by the addition of a single nucleotide per cycle (assuming the nucleotide to be added is complementary to a template nucleotide). The added nucleotides correspond to sequence that is complementary to at least a portion of the template.

In a preferred embodiment of the invention, inhibitory nucleotide analogs block further base addition by blocking the 3′ hydroxyl of the added base. Such 3′ blockers are cleavable such that the 3′ hydroxyl is regenerated for subsequent base addition. In a preferred embodiment, cleavage is chemical. For example, certain 3′ blockers of the invention have a disulfide that is cleaved by the addition of a reducing agent after incorporation and detection. Other analogs of the invention require a two-step process to regenerate the hydroxyl group, the first being a chemical cleavage and the second being a beta elimination that can proceed uncatalyzed or that can be aided by catalysis. While disulfide groups are preferred for their ease of removal, other chemically-labile groups can be used.

Analogs of the invention are preferably labeled as described below. Labels can be placed on the base portion of the molecule (e.g., at the 7-deaza position of the 5′ carbon of the base) or they can be attached to the reversible inhibitor at the 3′ hydroxyl. In the latter scenario, cleavage of the inhibitor results in cleavage of the label as well.

In one embodiment of the invention, after one or more primer extension steps, the template is removed from the duplex. The template is removed by any suitable means, for example by raising the temperature of the surface or the flow cell such that the duplex is melted, or by changing the buffer conditions to destabilize the duplex, or combination thereof. Methods for melting template/primer duplexes are well known in the art and are described, for example, in chapter 10 of Molecular Cloning, a Laboratory Manual, 3^{rd} Edition, J. Sambrook, and D. W. Russell, Cold Spring Harbor Press (2001), the teachings of which are incorporated herein by reference. The template is then removed from the surface, for example, by rinsing the surface with a suitable rinsing solution.

After removing the template, the extended primer used in the polymerization reaction remains on the surface. The 3′ terminus of the primer is then modified by addition of a short polynucleotide. The polynucleotide is added to the primer by enzymatic catalysis. A preferred enzyme is a ligase or a polymerase. Suitable ligases include, for example, T4 DNA ligase and T4 RNA ligase (such ligases are available commercially, from New England BioLabs (on the World Wide Web at NEB.com) and others capable of adding nucleotides to the 3′ terminus of the primer. In a preferred embodiment, a dephosphorylated polynucleotide is added to the primer. Methods for using ligases and dephosphorylating oligonucleotides are well known in the art.

If polymerization is used to add polynucleotides to the 3′ terminus of the primer, any suitable enzyme can be used. For example, a polymerase, such as poly(A) polymerase, including yeast poly(A) polymerase, commercially available from USB (on the World Wide Web at USBweb.com), terminal deoxyribonucleotidyl transferase (TdT), and the like are useful. The polymerases can be used according to the manufacturer's instructions.

Having been modified as described above, the primer is then used as a template for template-dependent sequencing-by-synthesis as described generally above.

The polynucleotide added to the primer is chosen such that it is complementary to a new primer (or at least a portion thereof). In a preferred embodiment, the polynucleotide is a homopolymer, such as oligo(dA), and the corresponding primer includes an oligo(dT) sequence. The complementary sequences are of a length suitable for hybridization. The added polynucleotide and its complementary new primer can be about 10 to about 100 nucleotides in length, and preferably about 50 nucleotides in length. The added polynucleotide and new primer can be of the same length or of different lengths. It is routine in the art to adjust primer length and/or oligonucleotide length to optimize hybridization.

Once a polynucleotide is added to the 3′ end of the primer and a new primer sequence is hybridized to the polynucleotide (or portion thereof), template-dependent sequencing-by-synthesis is conducted on the primer in the opposite direction of the original sequencing reaction (i.e., toward to surface to which the primer is bound).

After conducting the sequencing reaction back toward to the surface, the “new” extended primer can be melted off, leaving a template having the complementary sequence as the original template for optional resequencing in the 3′ to 5′ direction (i.e., toward the surface).

Sequencing and/or resequencing at least a portion of the complement of the original template increases the accuracy of the sequence information obtained from a given template by providing more than one set of sequence information to compare, for example, to a reference sequence. In another embodiment, the sequence initially obtained can be compared to the sequence obtained from the new template.

Sequencing methods of the invention preferably comprise template/primer duplex attached to a surface. Individual nucleotides herein added to the surface comprise a detectable label—preferably an optically-detectable label, such as a fluorescent label. Each nucleotide species can comprise a different label, or can comprise the same label. In a preferred embodiment, each duplex is individually optically resolvable in order to facilitate single molecule sequence discrimination. The choice of a surface for attachment of duplex depends upon the detection method employed. Preferred surfaces for methods of the invention include epoxide surfaces and polyelectrolyte multilayer surfaces, such as those described in Braslavsky, et al., supra. Surfaces preferably are deposited on a substrate that is amenable to optical detection of the surface chemistry, such as glass or silica.

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. Embodiments include the compounds of any of the formulae herein.

Polymerases useful in the invention include any nucleic acid polymerase capable of catalyzing a template-dependent addition of a nucleotide or nucleotide analog to a primer. Depending on the characteristics of the target nucleic acid, a DNA polymerase, an RNA polymerase, a reverse transcriptase, or a mutant or altered form of any of the foregoing can be used. According to one aspect of the invention, a thermophilic polymerase is used, such as ThermoSequenase®, 9°N™, Therminator™, Taq, Tne, Tma, Pfu, Tfl, Tth, Tli, Stoffel fragment, Vent™ and Deep Vent™ DNA polymerase.

Another aspect of the invention is a compound of any of the formulae herein for use in nucleic acid synthesis methods, or sequencing techniques as delineated herein.

Another aspect of the invention is the use of a compound of any of the formulae herein in the manufacture of a kit useful in nucleic acid synthesis methods, or sequencing techniques as delineated herein.

### BRIEF DESCRIPTION OF THE DRAWING

The FIGURE shows a schematic representation of one embodiment of the present invention.

### DETAILED DESCRIPTION

The invention provides molecules and methods to facilitate primer extension and nucleotide manipulation in sequencing techniques. While applicable to bulk sequencing methods, the invention is particularly useful in connection with single molecule sequencing methods. The invention provides nucleotide analogs that allow a single nucleotide to be added to a template/primer duplex at a time. The invention solves the problem of homopolymer run-through by allowing single nucleotide additions so that the number of bases in a homopolymeric stretch can be counted.

The invention also provides methods that utilize base-at-a-time analogs. Methods of the invention comprise the steps of exposing a duplex comprising a template and a primer to a polymerase and one or more nucleotide(s)/nucleotide analog(s) of the invention that temporarily inhibit subsequent base addition to the primer under conditions sufficient for template-dependent nucleotide addition to the primer. In one embodiment, the template is individually optically resolvable and added bases comprise an optically-detectable label. Any unincorporated labeled nucleotide(s)/nucleotide analog(s) is optionally washed way. Any nucleotide(s)/nucleotide analog(s) incorporated into the primer is identified by detecting the label associated with the incorporated nucleotide(s)/nucleotide analog(s). Inhibition is then removed, and the steps of exposing duplex to polymerase and another nucleotide(s)/nucleotide analog(s) comprising a detectable label and polymerizing, optional washing, and identification are repeated, thereby determining a nucleotide sequence. As a result of the exposing and polymerizing steps, the primer is extended by the addition of nucleotides that are complementary to the corresponding positions of the template.

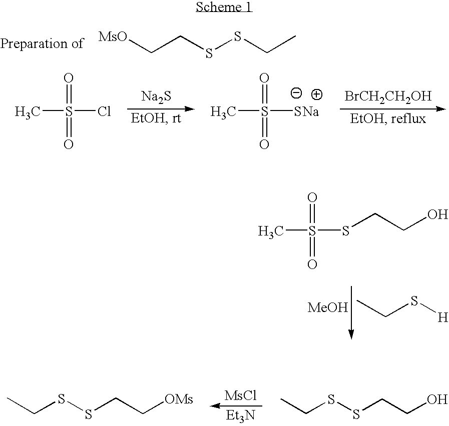

The Scheme 1 is a schematic representation of the preparation of an exemplary alkylating agent useful in making the compounds of the invention. In this embodiment, the 2-bromoethanol and ethylsulfide reagents depicted can be replaced with any suitable alkyl chain homologue to provide the desired alkyl chain length.

Scheme 2 is a schematic representation of the preparation of an exemplary protected nucleotide useful in the nucleic acid synthesis and sequencing methods of the invention. In this embodiment, Compound 4 can be used in the nucleotide extension of the primer with the 3′-hydroxy protected from further reaction, then mild deprotection with TCEP provides rapid, and efficient unmasking of the 3′-hydroxy group to allow further elaboration of the primer in the sequencing process. Alternatively, Compound 4 can be deprotected and elaborated to a compound of a formulae herein, or the TBS-protecting group can be interchanged with another group amenable to synthesis of a compound of a formulae herein.

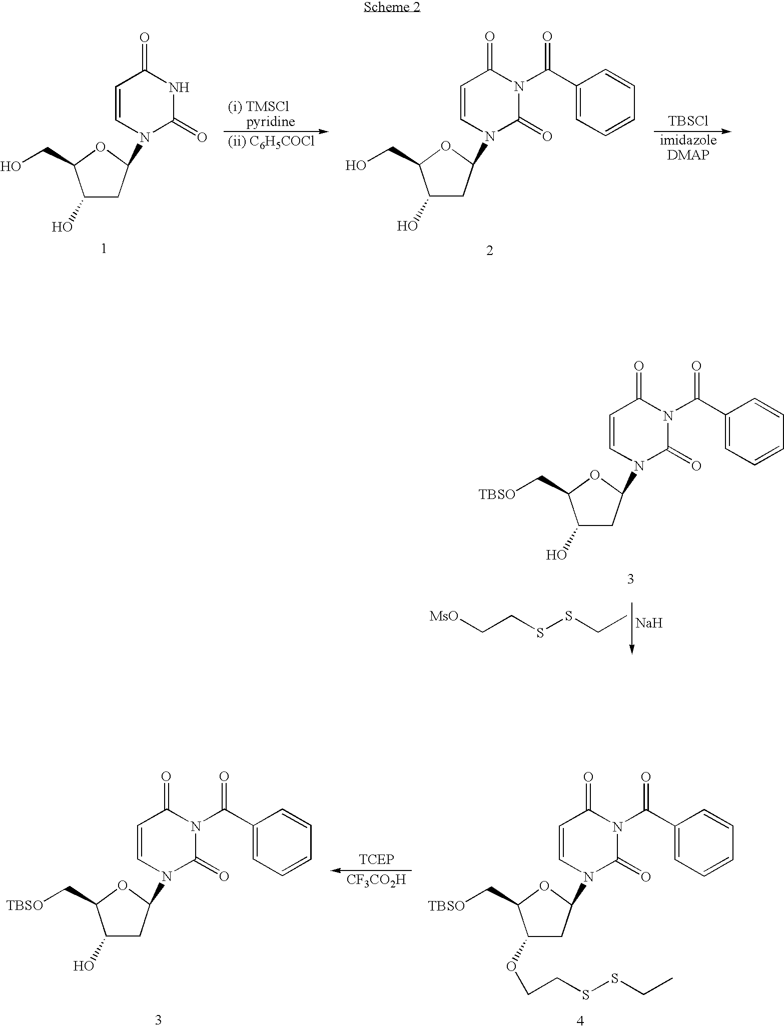

As is appreciated by the skilled artisan, the synthetic schemes herein are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof. Protecting groups as known in the art are described generally in T. H. Greene and P. G. M. Wuts, Protective Groups in Organic Synthesis, 3rd edition, John Wiley & Sons, New York (1999).

In a preferred use of analogs of the invention, direct amine attachment is used to attach primer or template to an epoxide surface. The primer or the template can comprise an optically-detectable label in order to determine the location of duplex on the surface. At least a portion of the duplex is optically resolvable from other duplex on the surface. The surface is preferably passivated with a reagent that occupies portions of the surface that might, absent passivation, fluoresce. Optimal passivation reagents include amines, phosphate, water, sulfates, detergents, and other reagents that reduce native or accumulating surface fluorescence. Sequencing is then accomplished by presenting one or more labeled nucleotide in the presence of a polymerase under conditions that promote complementary base incorporation in the primer. In a preferred embodiment, one base at a time (per cycle) is added and all bases have the same label. There is a wash step after each incorporation cycle, and the label is either neutralized without removal or removed from incorporated nucleotides and inhibition is removed. After the completion of a predetermined number of cycles of base addition, the linear sequence data for each individual duplex is compiled. Numerous algorithms are available for sequence compilation and alignment as discussed below.

In general, epoxide-coated glass surfaces are used for direct amine attachment of templates, primers, or both. Amine attachment to the termini of template and primer molecules is accomplished using terminal transferase. Primer molecules can be custom-synthesized to hybridize to templates for duplex formation.

A full-cycle is conducted as many times as necessary to complete sequencing of a desired length of template, or resequencing of the desired length of the template complementary sequence. Once the desired number of cycles is complete, the result is a stack of images represented in a computer database. For each spot on the surface that contained an initial individual duplex, there will be a series of light and dark image coordinates, corresponding to whether a base was incorporated in any given cycle. For example, if the template sequence was TACGTACG and nucleotides were presented in the order CAGU(T), then the duplex would be “dark” (i.e., no detectable signal) for the first cycle (presentation of C), but would show signal in the second cycle (presentation of A, which is complementary to the first T in the template sequence). The same duplex would produce signal upon presentation of the G, as that nucleotide is complementary to the next available base in the template, C. Upon the next cycle (presentation of U), the duplex would be dark, as the next base in the template is G. Upon presentation of numerous cycles, the sequence of the template would be built up through the image stack. The sequencing data are then fed into an aligner as described below for resequencing, or are compiled for de novo sequencing as the linear order of nucleotides incorporated into the primer.

The imaging system used in practice of the invention can be any system that provides sufficient illumination of the sequencing surface at a magnification such that single fluorescent molecules can be resolved.

General Considerations

A. Nucleic Acid Templates

Nucleic acid templates include deoxyribonucleic acid (DNA) and/or ribonucleic acid (RNA). Nucleic acid template molecules can be isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. Biological samples for use in the invention also include viral particles or samples prepared from viral material. Nucleic acid template molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid template molecules may also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA.

Nucleic acid obtained from biological samples typically is fragmented to produce suitable fragments for analysis. In one embodiment, nucleic acid from a biological sample is fragmented by sonication. Nucleic acid template molecules can be obtained as described in U.S. Patent Application 2002/0190663 A1, published Oct. 9, 2003, the teachings of which are incorporated herein in their entirety. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, individual nucleic acid template molecules can be from about 5 bases to about 20 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x-9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulfonate. It is contemplated also that urea may be added with or without another detergent or surfactant.

Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

B. Nucleotides

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. A nucleotide corresponds to a specific nucleotide species if they share base-complementarity with respect to at least one base.

Nucleotides for nucleic acid sequencing according to the invention preferably comprise a detectable label that is directly or indirectly detectable. Preferred labels include optically-detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dmethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

C. Nucleic Acid Polymerases

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J Biol. Chem. 256:3112), and archaeal DP1I/DP2 DNA polymerase II (Cann et al., 1998, Proc Natl Acad. Sci. USA 95:14250-->5).

While mesophilic polymerases are contemplated by the invention, preferred polymerases are thermophilic. Thermophilic DNA polymerases include, but are not limited to, ThermoSequenase®, 9Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347 (1975)).

D. Surfaces

In a preferred embodiment, nucleic acid template molecules are attached to a substrate (also referred to herein as a surface) and subjected to analysis by sequencing as taught herein. Nucleic acid template molecules are attached to the surface such that the template/primer duplexes are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methylmethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

In one embodiment, a substrate is coated to allow optimum optical processing and nucleic acid attachment. Substrates for use in the invention can also be treated to reduce background. Exemplary coatings include epoxides, and derivatized epoxides (e.g., with a binding molecule, such as streptavidin). The surface can also be treated to improve the positioning of attached nucleic acids (e.g., nucleic acid template molecules, primers, or template molecule/primer duplexes) for analysis. As such, a surface according to the invention can be treated with one or more charge layers (e.g., a negative charge) to repel a charged molecule (e.g., a negatively charged labeled nucleotide). For example, a substrate according to the invention can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Molecular Dynamics, Sunnyvale, Calif. In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase template molecule binding to the substrate or, at least, do not substantially impair template binding.

Various methods can be used to anchor or immobilize the primer to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11:107-115, 1986. A preferred attachment is direct amine bonding of a terminal nucleotide of the template or the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

E. Detection

Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

F. Analysis

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

In resequencing, a preferred embodiment for sequence alignment compares sequences obtained to a database of reference sequences of the same length, or within 1 or 2 bases of the same length, from the initially obtained sequence or the target sequence contained in a look-up table format. In a preferred embodiment, the look-up table contains exact matches with respect to the reference sequence and sequences of the prescribed length or lengths that have one or two errors (e.g., 9-mers with all possible 1-base or 2-base errors). The obtained sequences are then matched to the sequences on the look-up table and given a score that reflects the uniqueness of the match to sequence(s) in the table. The obtained sequences are then aligned to the reference sequence based upon the position at which the obtained sequence best matches a portion of the reference sequence. More detail on the alignment process is provided below in the Example.

### EXAMPLE

The 7249 nucleotide genome of the bacteriophage M13 mp18 was sequenced using single molecule methods of the invention. Purified, single-stranded viral M13 mp18 genomic DNA was obtained from New England Biolabs. Approximately 25 ug of M13 DNA was digested to an average fragment size of 40 bp with 0.1 U Dnase I (New England Biolabs) for 10 minutes at 37° C. Digested DNA fragment sizes were estimated by running an aliquot of the digestion mixture on a precast denaturing (TBE-Urea) 10% polyacrylamide gel (Novagen) and staining with SYBR Gold (Invitrogen/Molecular Probes). The DNase I-digested genomic DNA was filtered through a YM10 ultrafiltration spin column (Millipore) to remove small digestion products less than about 30 nt. Approximately 20 pmol of the filtered DNase I digest was then polyadenylated with terminal transferase according to known methods (Roychoudhury, R and Wu, R. 1980, Terminal transferase-catalyzed addition of nucleotides to the 3′ termini of DNA. Methods Enzymol. 65(1):43-62.). The average dA tail length was 50+/−5 nucleotides. Terminal transferase was then used to label the fragments with Cy3-dUTP. Fragments were then terminated with dideoxyTTP (also added using terminal transferase). The resulting fragments were again filtered with a YM10 ultrafiltration spin column to remove free nucleotides and stored in ddH₂O at −20° C.

Epoxide-coated glass slides were prepared for oligo attachment. Epoxide-functionalized 40 mm diameter #1.5 glass cover slips (slides) were obtained from Erie Scientific (Salem, N.H.). The slides were preconditioned by soaking in 3×SSC for 15 minutes at 37° C. Next, a 500 pM aliquot of 5′ aminated polydT(50) primer (polythymidine of 50 nucleotides in length with a 5′ terminal amine) is incubated with each slide for 30 minutes at room temperature in a volume of 80 ml. The resulting slides have primer attached by direct amine linkage to the epoxide. The slides are then treated with phosphate (1 M) for 4 hours at room temperature in order to passivate the surface. Slides re then stored in polymerase rinse buffer (20 mM Tris, 100 mM NaCl, 0.001% Triton X-100, pH 8.0) until they are used for sequencing.

For sequencing, the slides are placed in a modified FCS2 flow cell (Bioptechs, Butler, Pa.) using a 50 um thick gasket The flow cell is placed on a movable stage that is part of a high-efficiency fluorescence imaging system built around a Nikon TE-2000 inverted microscope equipped with a total internal reflection (TIR) objective. The slide is then rinsed with HEPES buffer with 100 mM NaCl and equilibrated to a temperature of 50° C. An aliquot of poly(dT50) template is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains M13 template/primer duplex. The temperature of the flow cell is then reduced to 37° C. for sequencing and the objective is brought into contact with the flow cell.

For sequencing, cytosine triphosphate, guanidine triphosphate, adenine triphosphate, and uracil triphosphate, each having a cyanine-5 label (at the 7-deaza position for ATP and GTP and at the C5 position for CTP and UTP (PerkinElmer)) and a 3′ blocking group comprising a ethyl dithio linkage are stored separately in buffer containing 20 mM Tris-HCl, pH 8.8, 10 mM MgSO₄, 10 mM (NH₄)₂SO₄, 10 mM HCl, and 0.1% Triton X-100, and 100 U Klenow exo⁻ polymerase (NEN). Sequencing proceeds as follows.

First, initial imaging is used to determine the positions of duplex on the epoxide surface. The Cy3 label attached to the M13 templates is imaged by excitation using a laser tuned to 532 nm radiation (Verdi V-2 Laser, Coherent, Inc., Santa Clara, Calif.) in order to establish duplex position. For each slide only single fluorescent molecules imaged in this step are counted. Imaging of incorporated nucleotides as described below is accomplished by excitation of a cyanine-5 dye using a 635 nm radiation laser (Coherent). 5 uM Cy5CTP is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 ul volumes each, followed by 150 mM HEPES/150 mM NaCl/pH 7.0 (“HEPES/NaCl”) (10 times at 60 ul volumes). An oxygen scavenger containing 30% acetonitrile and scavenger buffer (134 ul HEPES/NaCl, 24 ul 100 mM Trolox in MES, pH6.1, 10 ul DABCO in MES, pH6.1, 8 ul 2M glucose, 20 ul NaI (50 mM stock in water), and 4 ul glucose oxidase) is next added. The slide is then imaged (500 frames) for 0.2 seconds using an Inova301K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 ul) and HEPES/NaCl (60 ul). Next, the cyanine-5 label is cleaved off incorporated CTP by introduction into the flow cell of 50 mM TCEP for 5 minutes, after which the flow cell is rinsed 5 times each with SSC/HEPES/SDS (60 ul) and HEPES/NaCl (60 ul). The 3′ blocker is next cleaved by a two-step process that includes the addition of dithiothreitol (DTT) to cleave the disfulfide bond, followed by a beta elimination of the remaining ethylsulfhydryl group. The nucleotide is capped with 50 mM iodoacetamide for 5 minutes followed by rinsing 5 times each with SSC/HEPES/SDS (60 ul) and HEPES/NaCl (60 ul). The scavenger is applied again in the manner described above, and the slide is again imaged to determine the effectiveness of the cleave/cap steps and to identify non-incorporated fluorescent objects.

The procedure described above is then conducted 100 nM Cy5dATP, followed by 100 nM Cy5dGTP, and finally 500 nM Cy5dUTP. The procedure (expose to nucleotide, polymerase, rinse, scavenger, image, rinse, cleave, rinse, cap, rinse, scavenger, final image) is repeated exactly as described for ATP, GTP, and UTP except that Cy5dUTP is incubated for 5 minutes instead of 2 minutes. Uridine is used instead of Thymidine due to the fact that the Cy5 label is incorporated at the position normally occupied by the methyl group in Thymidine triphosphate, thus turning the dTTP into dUTP. In all 64 cycles (C, A, G, U) are conducted as described in this and the preceding paragraph.

Once the desired number of cycles are completed, the image stack data (i.e., the single molecule sequences obtained from the various surface-bound duplex) are aligned to the M13 reference sequence. The image data obtained can be compressed to collapse homopolymeric regions. Thus, the sequence “TCAAAGC” is represented as “TCAGC” in the data tags used for alignment. Similarly, homopolymeric regions in the reference sequence are collapsed for alignment.

The alignment algorithm matches sequences obtained as described above with the actual M13 linear sequence. Placement of obtained sequence on M13 is based upon the best match between the obtained sequence and a portion of M13 of the same length, taking into consideration 0, 1, or 2 possible errors. All obtained 9-mers with 0 errors (meaning that they exactly match a 9-mer in the M13 reference sequence) are first aligned with M13. Then 10-, 11-, and 12-mers with 0 or 1 error are aligned. Finally, all 13-mers or greater with 0, 1, or 2 errors are aligned.

The template fragments are removed by increasing the temperature of the flow cell above the melting temperature of the duplex, thereby releasing the template fragments from the duplexes. The free templates are removed from the flow cell by washing the flow cell, for example the flow cell can be rinsed 5 times each with SSC/HEPES/SDS (60 ul) and HEPES/NaCl (60 ul).

The primers are then modified by adding a polynucleotide sequence to the 3′ terminus of the primer. The oligonucleotide-modified primers are then used as the template in subsequent polymerization reactions. Free primer capable of hybridizing to the added oligonucleotide is added to the flow cell and incubated under conditions sufficient to allow hybridization between the added oligonucleotide portion of the template and the free primer. After incubation, the flow cell is rinsed with 1×SSC/HEPES/0.1% SDS followed by HEPES/NaCl. The resulting slide contains template/primer duplexes where the template comprises the original primer having M13 template complementary sequences added thereto and modified with an oligonucleotide. The temperature of the flow cell is then reduced to 37° C. for sequencing and the objective is brought into contact with the flow cell. The procedure (expose to nucleotide, polymerase, rinse, scavenger, image, rinse, cleave, rinse, cap, rinse, scavenger, final image) is repeated as described above.

Once the desired number of cycles is completed, the image stack data (i.e., the single molecule sequences obtained from the various surface-bound duplex) are aligned to the M13 reference sequence and/or are aligned to the sequence initially obtained as described above. The image data obtained can be compressed to collapse homopolymeric regions as described above.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed:

1. A molecule of formula (I): 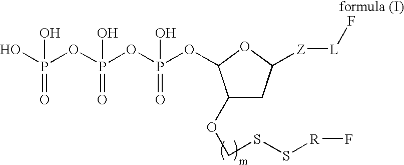
wherein,
Z is a purine, pyrimidine or analog thereof,
L is a linker;
Each F is independently an optically-detectable label;
R is alkyl; and
m is an integer greater than 1.

2. The molecule of claim 1, wherein the linker comprises an alkynyl group.

3. The molecule of claim 1, wherein the linker comprises the structure: 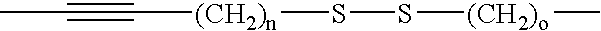
wherein,
n is an integer 1-7 inclusive; and
o is an integer 1-7 inclusive.

4. The molecule of claim 1, wherein each F is independently a fluorescent label.

5. The molecule of claim 1, wherein each F is independently cyanin-3 or cyanin-5.

6. The molecule of claim 1, wherein R is an alkyl having from about 1 to about 12 carbon atoms.

7. The molecule of claim 1, wherein the purine is adenine, guanine, or analog thereof.

8. The molecule of claim 1, wherein the pyrimidine is cytosine, thymidine, uracil, or analogs thereof.

9. A molecule of formula (II): 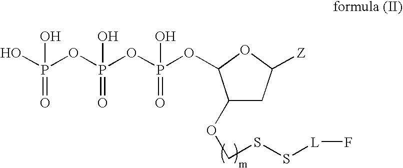
wherein,
Z is a purine, pyrimidine or analog thereof,
L is a linker;
F is an optically-detectable label; and
m is an integer greater than 1.

10. The molecule of claim 9, wherein the linker comprises an alkynyl group.

11. The molecule of claim 9, wherein the linker comprises the structure: 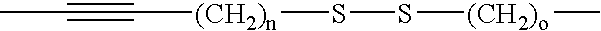
wherein,
n is an integer 1-7 inclusive; and
o is an integer 1-7 inclusive.

12. The molecule of claim 9, wherein F is a fluorescent label.

13. The molecule of claim 9, wherein F is cyanin-3 or cyanin-5.

14. The molecule of claim 9, wherein the purine is adenine, guanine, or analog thereof.

15. The molecule of claim 9, wherein the pyrimidine is cytosine, thymidine, uracil, or analogs thereof.

16. The molecule of claim 9, wherein m is 2.

17. The molecule of claim 1, wherein m is 2.

18. A method for sequencing a nucleic acid template comprising:
(a) exposing a nucleic acid duplex comprising a template nucleic acid hybridized to a primer nucleic acid to a plurality of molecules of a compound according to any of claims 1-17 under conditions that allow the molecule to be incorporated into the 3′-terminus of the primer and to engage in complementary base pairing with a nucleotide in the template.

19. The method of claim 18, further comprising:
(b) removing unincorporated molecules of the compound of any of claims 1-17; (c) observing a label associated with the compound of any of claims 1-17; (d) removing the label; (e) modifying the incorporated molecule to generate a free 3′-hydroxy group, and (f) repeating steps (a) to (e).

20. The method of claim 19, further comprising repeating step (f).

21. The method of claim 19, wherein step (b) comprises exposing the duplex to an agent capable of reducing disulfide bonds.

22. The method of claim 18, further comprising the step of identifying the molecule incorporated into the primer.

23. The method of claim 19, wherein step (d) comprises exposing the duplex to an agent capable of reducing disulfide bonds.

24. The method of claim 19, wherein step (e) comprises exposing the duplex to an agent capable of reducing disulfide bonds.

25. The method of claim 19, wherein steps (d) and (e) are performed simultaneously.

26. The method of claim 21, wherein the agent is tris(2-carboxyethyl)phosphine hydrochloride (TCEP-HCl).

27. The method of claim 19, wherein the reduction of the disulfide bond is performed at about pH 7.0 or greater.

28. The method of claim 19, wherein the reduction of the disulfide bond is performed at about pH 9.0 or greater.

29. The method of claim 19, wherein the reduction of the disulfide bond is performed at about 25° C. or greater.

30. The method of claim 19, wherein the reduction of the disulfide bond is performed at about 37° C. or greater.

31. The method of claim 19, wherein the reduction of the disulfide bond is performed at about 50° C. or greater.

32. A method for synthesizing a nucleic acid analog comprising contacting a nucleic acid sequence with a compound of formula (I) in claim 1 or formula (II) in claim 9.
